(19) United States
(12) Patent Application Publication
(10) Pub. No.: US 2011/0203277 A1
(43) Pub. Date: Aug. 25, 2011

(54) METHOD AND APPARATUS FOR PRODUCING LIQUID BIOFUEL FROM SOLID BIOMASS

(75) Inventors: Petri Kukkonen, Helsinki (FI); Pekka Knuuttila, Porvoo (FI); Pekka Jokela, Espoo (FI)

(73) Assignee: UPM-KYMMENE CORPORATION, Helsinki (FI)

(21) Appl. No.: 13/126,789

(22) PCT Filed: Oct. 30, 2009
(86) PCT No.: PCT/FI09/50874

§ 371 Date: Apr. 29, 2011

(30) Foreign Application Priority Data
Oct. 31, 2008 (FI) 20086032

(51) Int. Cl. F01K 7/38; F01K 11/02; C07C 1/04; B01J 12/00

(52) U.S. Cl. 60/653; 60/670; 518/702; 422/198

(57) ABSTRACT

The invention relates to producing liquid hydro carbonaceous product (1) from solid biomass (2). In the invention solid biomass (2) is gasified in a gasifier (6) to produce raw synthesis gas (3). The raw synthesis gas (3) is conditioned to purify the raw synthesis gas (3) to obtain purified synthesis gas (4), the conditioning comprising lowering the temperature of the raw synthesis gas (3) in a cooler (19) producing saturated steam (51). Then the purified gas (4) is subjected to a Fischer-Tropsch synthesis in a Fischer-Tropsch reactor (5) to produce liquid hydro carbonaceous product (1). In the invention the saturated steam (51) produced by the cooler (19) is further superheated in a superheating boiler (50) for producing superheated steam (52).

### FIELD OF THE INVENTION

The present invention relates to producing liquid biofuel from solid biomass according to the preamble of claims 1, 17. More particularly the present invention relates to a method and apparatus for producing liquid hydro carbonaceous product from solid biomass by gasifying solid biomass in a gasifier to produce raw synthesis gas, conditioning of the raw synthesis gas to purify the raw synthesis gas to obtain purified synthesis gas, the conditioning comprising lowering the temperature of the raw synthesis gas in a cooler producing saturated steam, subjecting the purified gas to a Fischer-Tropsch synthesis in a Fischer-Tropsch reactor to produce liquid hydro carbonaceous product and operating the superheating boiler substantially exclusively with one or more by-products generated in the method for producing liquid hydro carbonaceous product from solid biomass.

### BACKGROUND OF THE INVENTION

It is know to produce liquid fuels starting from solid feedstock that contains organic material. During the production the solid feedstock is gasified to convert it into raw synthesis gas. The formed raw synthesis gas is then purified into a purified synthesis gas. The purified synthesis gas in further converted into a liquid hydro carbonaceous product using Fischer-Tropsch-type synthesis. The thus formed liquid hydro carbonaceous product may be then upgraded to produce liquid biofuel. This kind of biomass to liquid processes are generally know for example from publications US 2005/0250862 A1 and WO 2006/043112.

The temperature of the raw synthesis gas coming from the gasification is generally at least about 700° C. or more. During the purification of the raw synthesis gas the temperature of the synthesis gas has to be lowered to a temperature needed for removing solid particles from the raw synthesis gas.

The lowering of the temperature of the raw synthesis gas is essential for purification steps, such as filtering step, water-gas-shift (WGS) step and scrubbing step, arranged downstream of the cooling step. The raw synthesis gas is cooled before conducting it into the filtering step, because if raw synthesis gas would be fed uncooled from the gasifier into a filter, the temperature of the raw synthesis gas could cause the particles removed from the raw synthesis gas to sintrate or clog to the filter. Furthermore the WGS reactor and scrubber are designed to operate at temperatures that are essential lower than about 700° C.

Accordingly, the temperature of the raw synthesis gas is lowered in a cooler during the purification of the raw synthesis gas. During cooling the temperature of the raw synthesis gas is lowered to between about 175 to 275° C., depending on the application. Cooler may comprise an evaporator or alternatively a feed water preheater and an evaporator. Thus during the cooling steam may be generated in the cooler.

The problem relating to the cooling is that the raw synthesis gas to be cooled consists mainly of hydrogen and carbon monoxide at reducing atmosphere. Because of the corrosive gas mixture of the raw synthesis gas the heat surfaces of the cooler may face metal dusting, as a consequence of which the cooler may produce only saturated steam, having temperature about 300 to 330° C. This kind of saturated steam cannot be utilized efficiently.

### BRIEF DESCRIPTION OF THE INVENTION

An object of the present invention is to provide a method and an apparatus so as to solve the above problems. The objects of the invention are achieved by a method according characterizing portion of claim 1. The method being characterized operating the superheating boiler substantially exclusively with one or more by-products generated in the method for producing liquid hydro carbonaceous product from solid biomass. The objects of the invention are further achieved by an apparatus according characterizing portion of claim 17. The apparatus being characterized in that the superheating boiler is arranged to be operated substantially exclusively with one or more by-products generated in the apparatus in the production of liquid hydro carbonaceous product from solid biomass.

According to the present invention the saturated steam generated in the cooling is further superheated in a superheating boiler for producing superheated steam, having temperature about 500 to 550° C. Thus the saturated steam generated in the cooler is converted in a form that may be utilized in a steam turbine or in the process of producing liquid biofuel from solid biomass itself.

In the present invention one or more by-products generated in producing liquid hydro carbonaceous product from solid biomass is utilized as fuel in the superheating boiler. In one embodiment tail gas generated in the Fischer-Tropsch synthesis is utilized as a fuel in the superheating boiler. In another embodiment of the present invention the raw synthesis gas is filtered in a filter to remove particles, such as ash and char, from the raw synthesis gas and at least part of the particles filtered in the filter is utlized as a fuel in the superheating boiler. In yet embodiment of the present invention the raw synthesis gas is purified by ultra purification for removing sulfur components, CO₂, H₂O, HCN, CH₃Cl, carbonyls, Cl and NOₓ sulfur from the raw synthesis gas and at least part of the by-product gas generated is utilized or destroyed in the superheating boiler. In one embodiment of the present invention the liquid hydro carbonaceous product obtained from Fischer-Tropsch synthesis is upgraded into biofuel and at least part of the by-product fractions generated in the upgrading is utilized as a fuel in the superheating boiler.

The advantage of the present invention is that superheating the saturated steam generated in the cooling step changes the saturated steam into a form that may be utilized further in the process of producing liquid biofuel from solid biomass or in a steam turbine. Thus, superheated steam produced in the superheating may enhance the total efficiency of the process for producing liquid biofuel. A further advantage of the present invention is that by-products originating from the process of producing liquid biofuel from solid biomass may be utilised in the superheating as fuel for the superheating boiler. The superheating boiler may thus be operated substantially exclusively with the by-products originating from the process of producing liquid biofuel from the solid biomass. Thus the synthesis gas or any other product gas or liquid generated in the process of producing liquid biofuel from the solid biomass is not used for superheating and the overall yield of the process is not reduced.

### BRIEF DESCRIPTION OF THE DRAWINGS

In the following the invention will be described in greater detail by means of preferred embodiments with reference to the attached drawings, in which

FIG. 1 shows a schematic flow chart of one embodiment of the present invention.

### DETAILED DESCRIPTION OF THE INVENTION

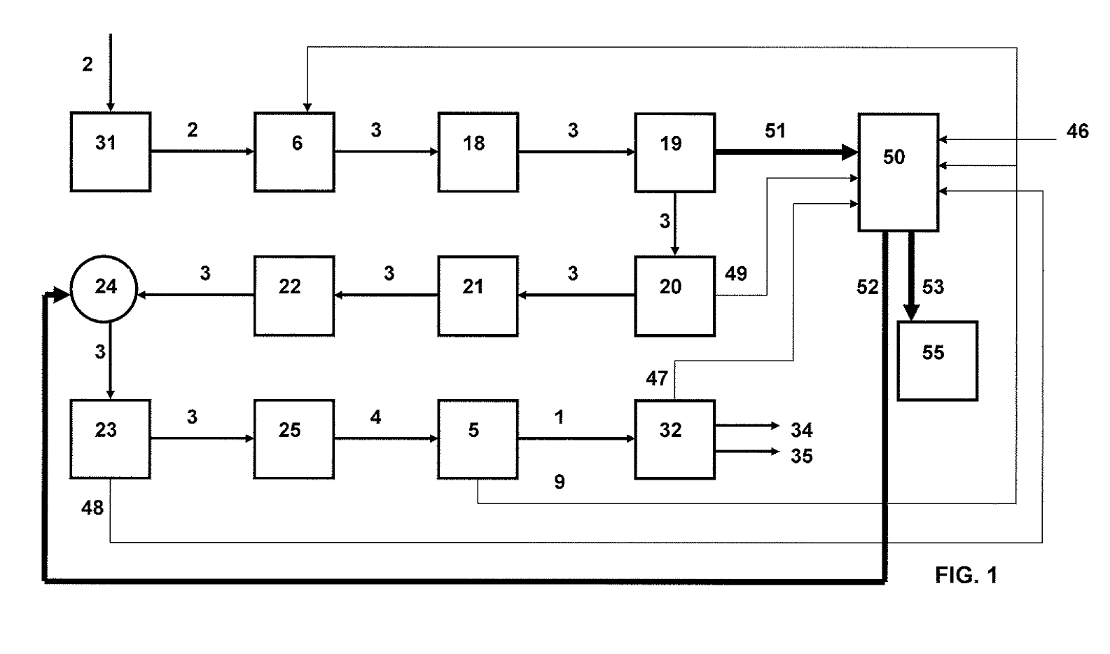

FIG. 1 is shows flow chart of one embodiment of a method and apparatus for producing liquid biofuel from solid biomass. However, it should be noted that the method and apparatus of the present invention comprises gasification of solid biomass 2 in a gasifier 6 into raw synthesis gas 3, conditioning the raw synthesis gas by conditioning means 18, 19, 20, 21, 22, 24, 23, 25 into purified synthesis gas 4 and subjecting the purified gas 4 to a Fischer-Tropsch synthesis in a Fischer-Tropsch reactor 5 to produce liquid hydro carbonaceous product 1, but the composition of conditioning steps and conditioning means may vary from one embodiment to another.

As shown in FIG. 1, the solid biomass 2 is fed to a gasifier 6 through solid biomass pretreatment and supply means 31. In this application the term solid biomass comprises substantially any kind of solid biomass that is suitable to be gasified. The solid biomass is typically selected from virgin and waste materials of plant, animal and/or fish industry, such as municipal waste, industrial waste or by-products, agricultural waste or by-products, waste or by-products of wood-processing industry, waste or by-products of food industry, marine plants and combinations thereof. The solid biomass may also comprise vegetable oils, animal fats, fish oils. Natural waxes and fatty acids, or the like that may also be alternatively in liquid form. The biomass pretreatment and supply means 31 may comprise crusher and/or dryer for crushing the solid biomass 2 and drying it to a moisture content less than 20%, preferably by thermal drying. The biomass 2 pretreatment and supply means 31 may further comprise a lock hopper for pressurising the solid biomass 2 at least to the pressure prevailing in the gasifier 6.

From the solid biomass pretreatment and supply means 31 the biomass 2 is fed to the gasifier 6. In the gasifier 6 the solid biomass 2 is gasified to produce raw synthesis gas 3 comprising carbon monoxide and hydrogen. In this context the raw synthesis gas means synthesis gas that in addition to carbon monoxide and hydrogen can contain impurities such as carbon dioxide (CO₂), methane (CH₄), water (H₂O), nitrogen (N₂), hydrogen sulfide (H₂S), ammonia (NH₃), hydrogen chloride (HCl), tar and small particles such as ash and soot. The gasifying step comprises at least partial combustion of the solid biomass 2 in a gasifier 6 to produce the raw synthesis gas 3. The gasifier 6 may be fluidized bed gasifier, for example a circulating fluidized bed reactor or a bubbling fluidized bed reactor. Oxygen and steam having temperature of about 200° C. and in addition possible also recycled tail gas 9 from the Fischer-Tropsch reactor 5 are used as fluidizing agents in the gasifier 6. The compounds of solid biomass 2 will react with the steam endothermically generating carbon monoxide and hydrogen and the compounds of the solid biomass 2 will react with the oxygen exothermically generating carbon monoxide, carbon dioxide and additional steam. The result of this is the raw synthesis gas 3. The gasifier may operate for example at 10 bar and 850° C.

From the gasifier 6 the raw synthesis gas 3 is fed to the conditioning or purification means to purify the raw synthesis gas obtained in the gasification. In a preferred embodiment the conditioning of the raw synthesis gas 3 comprises a sequence of conditioning steps and apparatuses in which various kind of conditioning of the raw synthesis gas is performed for purifying the raw synthesis gas 3 into a form suitable for a Fischer-Tropsch type synthesis. This means that for example the raw synthesis gas 3 is purified and the purified synthesis gas has a molar ratio of hydrogen to carbon monoxide between 2,5 to 1 and 0,5 to 1, preferably between 2,1 to 1 and 1,8 to 1, and more preferably about 2 to 1.

From the gasifier 6 the raw synthesis gas 3 is fed to a reformer 18 for catalytic treatment for converting tar and methane present in the raw synthesis gas 3 into carbon monoxide and hydrogen. Catalyst used in the reformer 18 may comprise for example nickel. Since tar and methane reforming are endothermic chemical reactions, and raw synthesis gas leaving the gasifier 6 is at too low temperature, the raw synthesis gas will be heated up before feeding it to the reformer 18, preferably by feeding oxygen into the raw synthesis gas. To prevent hotspots and ash melting, oxygen will be fired together with steam and recirculated FT tail gas. Thus the temperature of the raw synthesis gas is for example 900° C. before the raw synthesis gas flows into the reformer.

Between the gasifier 6 and the reformer 18 there may also be one or more particle separation steps for removing particles such as ash, char and bed material from the raw synthesis gas 3. The particle separation steps are performed preferably with one or more cyclones (not shown).

After the reformer 18 the raw synthesis gas 3 is fed to a subsequent conditioning step in which it is fed to a cooler 19 for lowering the temperature of the raw synthesis gas 3. During cooling the temperature of the raw synthesis gas 3 is lowered to between about 175 to 275° C., preferably to about 250° C., depending on the application. Cooler 19 may comprise an evaporator or alternatively a feed water preheater and an evaporator. Thus during the cooling steam is generated in the cooler 19. The raw synthesis gas 3 to be cooled consists mainly of hydrogen and carbon monoxide at reducing atmosphere. Because of the corrosive gas mixture of the raw synthesis gas 3 the heat surfaces of the cooler 19 may face metal dusting, as a consequence of which the temperature of the cooler 19 must be maintained in a range below the metal dusting temperature. Because of this, the cooler 19 may produce only saturated steam, having temperature for example about 300 to 330° C., at high pressure, such as 115 bar.

The cooling of the raw synthesis gas is essential for the next conditioning step, the filtering step following the cooling step. The raw synthesis gas 3 has to be cooled before conducting it into the filtering step, because if raw synthesis gas is fed uncooled from the gasifier 6 into a filter 20, the temperature of the raw synthesis gas 3 could cause the particles removed from the raw synthesis gas 3 to sintrate or clog to the filter 20 used in the filtering step. The filter 20 comprises preferably a metallic or sinter candle filter. The filter cake will be removed from the filter elements by repeating nitrogen or CO pressure shock. In the filter 20 solid particles, such as ash, soot, char and entrained bed materials are removed from the raw synthesis gas 3.

The conditioning of the raw synthesis gas 3 comprises preferably also a step for adjusting the molar ratio of hydrogen and carbon monoxide by a water-gas-shift reaction in a water-gas-shift (WGS) reactor 21 to between 2,5 to 1 and 0,5 to 1, preferably between 2,1 to 1 and 1,8 to 1, and more preferably about 2 to 1. The WGS reactor 21 is located downstream of the filter 20 and thus the filtered raw synthesis gas 3 is fed to the WGS reactor 21, as shown in FIG. 1.

The raw synthesis gas 3 is preferably further conditioned in a scrubber 22 to remove remaining solids, residual tar components and also HCl, NH₃ and other components from the raw synthesis gas 3 by scrubbing. The scrubber 22 is may located downstream of the WGS reactor 2, preferably such that raw synthesis gas 3 is fed from the WGS reactor 21 to the scrubber 22.

The conditioning of the raw synthesis gas 3 may also comprise ultra purification means 23 for cleaning of the raw synthesis gas. The ultra purification means removes sulfur components, such as H₂S, CO₂ (carbon dioxide), H₂O (water), HCN (hydrogen cyanide), CH₃Cl (methyl chloride), carbonyls, Cl (chloride) and NOₓ (nitrogen oxide) from the raw synthesis gas 3. Preferably the raw synthesis gas 3 is fed from the scrubber 22 to the ultra purification means 23. The ultra purification may be performed with physical cleaning process in which methanol or dimethyl ether is used a solvent at 30 to 40 bar pressure and cryogenic temperatures −25° to −60° C. Alternatively the ultra purification may be performed with chemical cleaning process in which the raw synthesis gas is chemically washed, for example with amine.

Before ultra purification means 23 the pressure of the raw synthesis gas 3 is raised in a compressor 24 to about 30 to 40 bar, such that the raw synthesis gas 3 entering the ultra purification means is already at a suitable pressure.

The conditioning may also comprise conditioning step comprising a guard bed reactor 25 in which possible residual sulfur components are removed from the raw synthesis gas 3. The guard bed reactor 25 comprises ZnO catalyst and active carbon. Preferably the guard bed reactor 25 is located downstream of the ultra purification means 23.

The conditioning of the raw synthesis gas 3 may comprise all the above mentioned steps and apparatuses or it may comprise only some of the steps and apparatuses described above. Furthermore, the conditioning means and steps may also comprise some additional conditioning steps and apparatuses that are not described. The sequence of the conditioning steps and apparatuses is preferably the above described, but it may also in some cases be different.

From the conditioning means, and in this case from the guard bed reactor 25, the purified synthesis gas 4, obtained from the raw synthesis gas 3 by the conditioning means, is fed to the Fischer-Tropsch reactor 5 for conducting the Fischer-Tropsch synthesis for the purified synthesis gas 4. In the Fischer-Tropsch synthesis carbon and hydrogen monoxide are converted into liquid hydrocarbons of various forms by catalyzed chemical reaction. The principal purpose of this process is to produce a synthetic petroleum substitute product, a liquid hydro carbonaceous product 1. The desired fuel component is diesel fraction and as a by-product also Naphta is produced. Fischer-Tropsch reactor 5 operates typically at a temperature of 200 to 220° C. Process includes an internal cooling and the produced heat can be utilized as low pressure steam. The Fischer-Tropsch synthesis produces also so called tail gas 9 as a by-product.

The liquid hydro carbonaceous product 1 may further be fed from the Fischer-Tropsch reactor 5 product upgrade section 32 where the he liquid hydro carbonaceous products 1 will be first flashed to separate the light hydro carbons from the product stream. The flashed product stream will be hydro cracked to maximize the diesel fraction. Hydro isomerisation will decrease the cloud point of the diesel fraction enabling usage of the diesel product in cold climates. In the distillation process, the heavy fractions are separated and circulated back to hydro cracking and hydro isomerisation section. Distillation also separates the final end products, diesel fractions 34 and naphtha fractions 35 from each other. The product upgrade may also separate some by-product fractions 47 from diesel and naphtha fractions 34, 35.

As described above, the temperature of the raw synthesis gas 3 or the purified synthesis gas 4 has to be lowered in a cooler during the conditioning of the synthesis gas because of the operating limits of the conditioning means and Fischer-Tropsch reactor 5. The cooler 19 is preferably located to the conditioning means and more preferably downstream of reformer 18 and prior to filter 20. As mentioned earlier, the cooler 20 comprises an evaporator or alternatively a feed water preheater and an evaporator. Thus during the cooling steam may be generated in the cooler 20. During cooling the temperature of the raw synthesis gas is lowered to between about 175 to 275° C., depending on the application. Typically the temperature of the raw synthesis gas 3 is lowered to about 250° C. Cooler 19 produces high pressure saturated steam 51 having preferably temperature about 300 to 330° C. and pressure about 100 to 130 bars. Typically the saturated steam 51 is at temperature about 320° C. and at pressure 115 bar.

According to the present invention the high pressure saturated steam 51 is fed from the cooler 19 to a superheating boiler 50 for producing superheated steam 52, 53. The superheating boiler 50 may be any known type superheating boiler that is suitable for superheating steam. Superheating boiler is a combustion apparatus which is equipped with a superheater for superheating the saturated steam circulating in the superheater tubing. As fuel for the combustion apparatus can be used different types of fuels. The superheated steam 52, 53 leaving the superheating boiler 50 is typically at temperature between 500 to 550° C., preferably 510° C., and at pressure about 100 to 130 bars, preferably at pressure 115 bar. This way the saturated steam from the cooler 19 may be converted into a form that may be utilized further in the method for producing liquid hydro carbonaceous product 1 or for producing energy.

The superheated steam 53 may further be fed to a steam turbine 55 for producing electrical energy. In this application, superheating boiler 50 is operatively connected to a steam turbine 55 for utilizing the superheated steam 53 in the steam turbine 55. If the apparatus for producing the liquid carbonaceous product 1 is located in connection with an industrial plant or at site of a mill, such as forest industry plant, the superheated steam 53 may be used in a steam turbine already existing. The forest industry plant may be a sawmill, cellulose mill, papermill comprising steam producing boiler(s), such as recovery boiler, power boiler, waste heat boiler that produce steam for a turbine. In that case thermal power corresponding amount of thermal power of the superheated steam 53 utilized in the steam turbine 55 may be saved in the existing boilers(s) of the forest industry plant. Thus the consumption of fuel may decrease.

Alternatively or additionally superheated steam 52 obtained from the superheating boiler 50 may be utilized for pressurising the raw synthesis gas 3 or the purified synthesis gas 4 before supplying it into the Fischer-Tropsch reactor 5. Thus the superheated steam 52 may also be fed from the superheating boiler 50 to the compressor 24, as is shown in FIG. 1. Thus the superheating boiler 50 is operatively connected to compressor 24 for utilizing the superheated steam 52 to pressurise the raw synthesis gas 3 before supplying it into the Fischer-Tropsch reactor 5.

In the present invention one or more by-products generated in the method or process for producing liquid hydro carbonaceous product 1 from solid biomass 2 is used as fuel in the superheating boiler 50. According to the present invention one or more by-products are used substantially exclusively for operating the superheating boiler 50.

As described above, tail gas is generated in the Fischer-Tropsch synthesis in the Fischer-Tropsch reactor 5. This tail gas 9 is very pure and contains combustible components. The main combustible components of the tail gas 9 are carbon monoxide, hydrogen, and hydrocarbons C1-C5. Furthermore, mass and energy calculations of the method for producing liquid hydro carbonaceous product 1 from solid biomass 2 indicate that the thermal power for superheating the saturated steam 51 generated in cooler 19 and the thermal power of the tail gas 9 correspond substantially to each other. Thus the tail gas 9 can be used as fuel for the superheating boiler 50 and it may be fed to the superheating boiler 50 with tail gas supply means. Some of the tail gas 9 may also be recirculated to the gasifier 6. The tail gas supply means comprise pipes and possible valves or the like for conducting the tail gas 9 from the Fischer-Tropsch reactor 5 to the superheating boiler 50.

Also at least part of the material filtered in the filter 20 may be utilized in the superheating boiler 50 as a fuel. The particles filtered from the raw synthesis gas 3 in the filter 20 comprise typically ash, soot and char. The ash comprises a lot of carbon, typically about 35 to 45%. Therefore ash 49 may be fed by particle supply means from the filter 20 to the superheating boiler 50 to be used as fuel for superheating the saturated steam 51. The particle supply means comprise pipes, conveyors or the like for conducting the ash 49 from the filter 20 to the superheating boiler 50.

The ultra purification means 23 generates a H₂S rich by-product gas 48 that contains also other sulfur components, CO₂ (carbon dioxide), H₂O (water), HCN (hydrogen cyanide), CH₃Cl (methyl chloride), carbonyls, Cl (chloride) and NOₓ (nitrogen oxide) as a result of the purification of the raw synthesis gas. This by-product gas 48 may in some embodiments be fed by ultra purification by-product supply means to the superheating boiler 50 to by used as fuel in it. In the same time, or alternatively, the superheating boiler 50 is also capable to destroy the H₂S rich gas 48 originated from the ultra purification 23. This H₂S rich gas 48 may not in all cases provide any additional fuel capacity in the superheating boiler 50 but this provides an alternative process step to destroy the odorous gas stream 48. If this H₂S containing, odorous gas 48 is burned in superheating boiler 50 the produced flue gases must be cleaned, for example by scrubbing to remove sulphur oxide components. Also the burner in the superheating boiler needs to be designed as low NOₓ burner to get the NOx content of the flue gases below the NOₓ emission levels. The by-product supply means may comprise pipes, valves or the like for conducting the by-product gases 48 from the ultra purification 23 to the superheating boiler 50.

In the product upgrade section 32 by-product fractions 47 may be generated in addition to diesel fractions 34 and naphtha fractions 35. These fractions contain gaseous or liquid light weight hydrocarbons. Also at least part of the product upgrade by-product fractions 47 may be fed with product upgrade by-product supply means to the superheating boiler 50 to be used as a fuel for superheating the saturated steam 51. The product upgrade by-product supply means may comprise pipes, valves or the like for conducting the by-product fractions 47 from the ultra purification 23 to the superheating boiler 50.

The superheating boiler 50 is preferably also arranged to use light fuel oil and/or natural gas as a support fuel 46 in the superheating boiler 50 for example for adjusting or controlling the operation of the superheating boiler. The support fuel 46 may also be utilized for start up. The tail gas 9 produced in the process provides substantially the same amount of fuel power as needed for operating the superheating boiler 50 in normal operating conditions. When necessary 15% or less of the total fuel power of the superheating boiler 50 may be supplied as separate support fuel 46 for adjusting or controlling the operation of the superheating boiler 50. In a preferred embodiment 10% or less, and more preferably 5% or less of the total fuel power of the superheating boiler 50 may be supplied to the superheating boiler 50 as separate support fuel 46. According to the present invention, no synthesis gas or other product gas or liquid is used to operate the superheating boiler 50. Therefore using the superheating boiler 50 does not decrease the yield of the liquid bio-fuel from the process.

Accordingly, the present invention provides that one or more by-products 9, 47, 48, 49, generated in a process for producing liquid biofuel from solid biomass 2 may be used, as fuel in a superheating boiler 50 for superheating the saturated steam 51 originated from the cooling step. The saturated steam originating from cooling the raw synthesis gas 3 in a process for producing liquid biofuel from solid biomass 2 may also be used for pressurising the purified synthesis gas 4 in a compressor before supplying it into the Fischer-Tropsch reactor 5. Therefore the energy efficiency of the method and apparatus for producing liquid hydro carbonaceous product 1 from biomass or the energy efficiency of an industrial plant having integrated apparatus for producing liquid hydro carbonaceous product 1 from biomass may be enhanced.

In one embodiment of the present invention feed water having temperature of 103° C. is supplied from the main boiler to the cooler 19. In the cooler 19 the feed water is vaporized as it receives thermal energy from the purified synthesis gas 4. The vaporized feed water attains temperature of 323° C. forms saturated steam 51. The saturated steam 51 is supplied to the superheating boiler 50 in which it is superheated to form superheated steam having temperature of 510° C. In the superheating boiler, tail gas is used as fuel. Support fuel 46 may be used in the superheating boiler 50 for adjusting the operation of the superheating boiler 50 to eliminate variations in the tail gas production.

It will be obvious to a person skilled in the art that, as the technology advances, the inventive concept can be implemented in various ways. The invention and its embodiments are not limited to the examples described above but may vary within the scope of the claims.

### LIST OF REFERENCE NUMERALS

1 Liquid hydro carbonaceous product
2 Solid biomass
3 Raw synthesis gas
4 Purified synthesis gas
5 Gasifier
9 Tail gas
18 Reformer
19 Cooler
20 Filter
21 Water Gas Shift (WGS) reactor
22 Scrubber
23 Ultra purification means
24 Compressor
25 Guard bed reactor
31 Solid biomass pre-treatment and supply means
32 Product upgrade means
34 Diesel fraction
35 Naphta fraction
46 Support fuel
47 Product upgrade by-product fractions
48 Ultra purification by-product gas
49 Filtered ash
50 Superheating boiler
51 Saturated steam
52 Superheated steam to steam to compressor
53 Superheated steam to steam turbine
55 Steam turbine

1.-30. (canceled)

31. A method for producing liquid hydro carbonaceous product from solid biomass, the method comprising:
gasifying solid biomass in a gasifier to produce raw synthesis gas;
conditioning of the raw synthesis gas to purify the raw synthesis gas to obtain purified synthesis gas, the conditioning comprising lowering the temperature of the raw synthesis gas in a cooler producing saturated steam;
subjecting the purified gas to a Fischer-Tropsch synthesis in a Fischer-Tropsch reactor to produce liquid hydro carbonaceous product;
superheating the saturated steam produced by the cooler in a superheating boiler for producing superheated steam by feeding the saturated steam to the superheating boiler prior to an utilization of said saturated steam; and
operating the superheating boiler substantially exclusively with one or more by-products generated in the method for producing liquid hydro carbonaceous product from solid biomass.

32. A method according to claim 31, comprising supplying tail gas generated in the Fischer-Tropsch synthesis to the superheating boiler to be used as a fuel in the superheating boiler.

33. A method according to claim 31, comprising conditioning of the raw synthesis gas by filtering the raw synthesis gas in a filter to remove particles, such as ash, from the raw synthesis gas.

34. A method according to claim 33, comprising supplying ash, comprising char, collected in the filter to the superheating boiler to be used as a fuel in the superheating boiler.

35. A method according to claim 31, comprising conditioning the raw synthesis gas by ultra purification for removing sulfur components, CO₂, H₂O, HCN, CH₃Cl, carbonyls, Cl and NOₓ from the raw synthesis gas.

36. A method according to claim 35, comprising supplying by-product gas generated in the ultra purification to the superheating boiler to be used as a fuel in the superheating boiler.

37. A method according to claim 35, comprising supplying H₂S rich by-product gas generated in the ultra purification to the superheating boiler to be destroyed in the superheating boiler.

38. A method according to claim 31, comprising product upgrading for upgrading the liquid hydro carbonaceous product obtained from Fischer-Tropsch synthesis to at least one diesel fraction and at least one naptha fraction.

39. A method according to claim 38, comprising supplying by-product fractions of the liquid hydro carbonaceous product generated in the product upgrading to the superheating boiler to be used as a fuel in the superheating boiler.

40. A method according to claim 31, comprising using separate support fuel for adjusting the operation of the superheating boiler.

41. A method according to claim 40, comprising using light fuel oil and/or natural gas as a support fuel in the superheating boiler.

42. A method according to claim 40, comprising supplying 15% or less of the total fuel power as support fuel in the superheating boiler.

43. A method according to claim 40, comprising supplying 10% or less of the total fuel power as support fuel in the superheating boiler.

44. A method according to claim 40, comprising supplying 5% or less of the total fuel power as support fuel in the superheating boiler.

45. A method according to claim 31, comprising utilizing the superheated steam in a steam turbine.

46. A method according to claim 31, comprising utilizing the superheated steam for pressurising the raw synthesis gas or the purified synthesis gas before supplying it into the Fischer-Tropsch reactor.

47. An apparatus for producing liquid hydro carbonaceous product from solid biomass, the apparatus comprising
a gasifier for gasifying solid biomass to produce raw synthesis gas;
conditioning means for conditioning the raw synthesis gas to obtain purified synthesis gas, the conditioning means comprising a cooler for lowering the temperature of the raw synthesis gas, the cooler being arranged to generate saturated steam;
a Fischer-Tropsch reactor for subjecting the purified gas to a Fischer-Tropsch synthesis to produce liquid hydro carbonaceous product; and
a superheating boiler for producing superheated steam from the saturated steam prior to an utilization of said saturated steam, wherein
the superheating boiler is arranged to be operated substantially exclusively with one or more by-products generated in the apparatus in the production of liquid hydro carbonaceous product from solid biomass.

48. An apparatus according to claim 47, wherein the apparatus comprises tail gas supply means for supplying tail gas generated in the Fischer-Tropsch synthesis to the superheating boiler to be used as a fuel in the superheating boiler.

49. An apparatus according to claim 47, wherein the conditioning means comprise a filter for filtering the raw synthesis gas in a filter to remove particles, such as ash, from the raw synthesis gas, and that apparatus further comprises particle supply means for supplying at least part of the particles filtered in the filter to the superheating boiler to be used as a fuel in the superheating boiler.

50. An apparatus according to claim 47, wherein the conditioning means comprise ultra purification means for removing sulfur components, CO₂, H₂O, HCN, CH₃C1, carbonyls, Cl and NOₓ from the raw synthesis gas, and that that apparatus further comprises ultra purification by-product supply means for supplying at least part of the by-product gas generated in the ultra purification to the superheating boiler.

51. An apparatus according to claim 50, wherein the ultra purification by-product supply means are arranged to supply H₂S rich by-product gas generated in the ultra purification to the superheating boiler to be destroyed in the superheating boiler.

52. An apparatus according to claim 47, wherein conditioning means comprises product upgrading means for upgrading the liquid hydro carbonaceous product obtained from Fischer-Tropsch synthesis, and that the apparatus further comprise product upgrade by-product supply means for supplying at least part of the by-product fractions generated in the product upgrading to the superheating boiler to be used as a fuel in the superheating boiler.

53. An apparatus according to claim 47, wherein the superheating boiler is arranged to use support fuel for adjusting the operation of the superheating boiler.

54. An apparatus according to claim 53, wherein 15% or less of the total fuel power is arranged to be supplied to the superheating boiler as support fuel.

55. An apparatus according to claim 54, wherein 10% or less of the total fuel power is arranged to be supplied to the superheating boiler as support fuel.

56. An apparatus according to claim 55, wherein 5% or less of the total fuel power is arranged to be supplied to the superheating boiler as support fuel.

57. An apparatus according to claim 47, wherein the superheating boiler is arranged to use light fuel oil and/or natural gas as a support fuel in the superheating boiler.

58. An apparatus according to claim 47, wherein the superheating boiler is operatively connected to a steam turbine for utilizing the superheated steam in the steam turbine.

59. An apparatus according to claim 47, wherein the superheating boiler is operatively connected to compressor for utilizing the superheated steam to pressurise the raw synthesis gas to the purified synthesis gas before supplying it into the Fischer-Tropsch reactor.

60. An apparatus according to claim 47, wherein it is an integral part of an industrial plant having a steam turbine.
